(19) United States
(12) Patent Application Publication
(10) Pub. No.: US 2021/0255194 A1
(43) Pub. Date: Aug. 19, 2021

(54) METHODS FOR IDENTIFYING AND ANALYZING AMINO ACID SEQUENCES OF PROTEINS

(71) Applicant: Oncobiologics, Inc., Cranbury, NJ (US)

(72) Inventors: Xiaoyao XIAO, Cranbury, NJ (US); Linghui LI, Cranbury, NJ (US)

(21) Appl. No.: 16/072,989

(22) PCT Filed: Feb. 3, 2017
(86) PCT No.: PCT/US2017/016549

§ 371 Date: Jul. 26, 2018

Related U.S. Application Data
(60) Provisional application No. 62/291,216, filed on Feb. 4, 2016.

(51) Int. Cl. G01N 33/68; G01N 30/72

(52) U.S. Cl. CPC G01N 33/6818; G01N 30/72; G01N 33/6848

(57) ABSTRACT

The disclosure provides methods for determining the biosimilarity of a test protein in relation to a target biologic in which the test protein is digested by two distinct proteases, the resultant digested peptide fragments are analyzed by column chromatography-tandem mass spectrometry to achieve 100% of the amino acid sequence coverage and 100% amino acid sequence accuracy of the test protein.

### RELATED APPLICATIONS

This application is a National Stage Application, filed under 35 U.S.C 371, of International Application No. PCT/US2017/016549, filed on Feb. 3, 2017, which claims priority to U.S. Patent Application No. 62/291,216, filed Feb. 4, 2016, the contents of each of these applications is incorporated by reference in its entirety.

### INCORPORATION OF SEQUENCE LISTING

The contents of the text file named “ONBI-008N01USSequence-Listing.txt”, which was created on Jul. 24, 2018 and is 81.7 KB in size, are hereby incorporated by reference in their entirety.

### FIELD OF THE DISCLOSURE

The disclosure relates generally to improved protein sequencing methods that use a reduced incubation time for protease digestion of denatured protein and includes an increased aqueous mobile phase during column chromatography and tandem mass spectrometry (LC-MS/MS) analysis to increase sequence coverage and accuracy up to 100%, as well as improved protein sequencing methods for use in development of therapeutic recombinant proteins and quality control analysis for the manufacture of approved biologics.

### BACKGROUND

Recombinant proteins including recombinant monoclonal antibodies (mAbs) and recombinant versions of natural proteins have been used as reagents for biomedical research, as well as diagnostic and therapeutic agents for humans. One example of recombinant proteins includes biosimilar molecules (also referred to as “biologics”). In order to be approved for use as therapeutic agents for humans, biosimilar molecules must be shown to have an identical amino acid sequence and be very nearly similar in posttranslational modifications, e.g., have “sameness”, to the parent innovator biologic product. Assessing the sameness of a biosimilar molecule is critical because recombinant proteins are complex in nature. Recombinant proteins are engineered using genetically-modified, living organisms (e.g., bacteria, yeast, animal or human cell lines). The living organisms produce recombinant proteins that are long chain amino acids and/or modified amino acids folded by complex mechanisms. Consequently, recombinant proteins exhibit high molecular complexity and are highly sensitive to changes in the manufacturing process.

The specificity and effector function of a recombinant protein is highly dependent on the amino acid sequence and the presence or absence of specific modifications. Accordingly, DNA sequencing is routinely used to initially characterize biologics, such as monoclonal antibodies. However, protein-level rearrangements such as subsequent mutations and posttranslational modifications (PTMs) of recombinant proteins, e.g., a monoclonal antibody, are recognized by analysis at the protein level because such rearrangements can only be revealed by protein level analysis. Therefore, amino acid sequencing of monoclonal antibodies is required when the cDNA or the original cell line for the antibody is not available, or when characterization of an amino acid sequence is necessary to verify similarity of the recombinant antibody for approved use as a therapeutic agent, as well as for quality control during manufacture.

Despite the importance of sequence identification of amino acids in proteins, no methods have been developed for sequencing unknown proteins that provide a high level (100%) of sequence accuracy and coverage. Sequencing recombinant proteins in particular remains a challenge. Two general approaches are used for sequencing proteins using mass spectrometry. In the first, intact proteins are ionized and then introduced to a mass analyzer for mass measurement and tandem mass spectrometry (MS/MS) analysis. This approach is referred to as “top-down” proteomics. In the second, proteins are enzymatically digested into smaller peptides using a protease such as trypsin. Subsequently, the peptides are introduced into a mass spectrometer and identified by peptide mass fingerprinting or tandem mass spectrometry (MS/MS). This latter approach is called “bottom-up” proteomics and uses identification at the peptide level to infer the existence of proteins. Bottom-up proteomics is a preferred process for identifying proteins and characterizing their amino acid sequences, as well as PTMs.

One well-known method of bottom-up proteomics is Edman degradation. In this method, the amino-terminal residue is labeled and cleaved from a peptide without disrupting the peptide bonds between other amino acid residues. Because Edman degradation proceeds from the N-terminus of the protein, it is unreliable if the N-terminal amino acid has been chemically modified or if it is concealed within the body of the native protein. It also requires guesswork or a separate procedure to determine the positions of disulfide bridges, as well as peptide concentrations of 1 picomolar or above, for discernible results. Consequently, the Edman process is unsuitable for sequencing proteins longer than 50 amino acids or proteins with PTMs.

Mass spectrometry-based methods characterize a protein by assembling tandem mass (MS/MS) spectra of overlapping peptides generated from multiple proteolytic digestions of the protein. Each tandem mass (MS/MS) spectrum covers only a short peptide of the target protein. Thus, the key to high coverage protein sequencing is to find spectral pairs from overlapping peptides in order to assemble tandem mass spectrometry (MS/MS) spectra to long ones. However, overlapping regions of peptides may be too short to be confidently identified. Further, automated de novo sequencing methods that rely on interpreting individual tandem mass spectrometry (MS/MS) spectra are limited because these methods typically cannot reconstruct long (8+ amino acid) sequences without misidentifying 1 in 5 amino acids on average. Advances in de novo peptide sequencing have improved sequencing accuracy to over 95%, but at limited sequence coverage, e.g., only 55% sequence coverage. All current per-spectrum de novo sequencing strategies face a tradeoff between sequencing accuracy versus coverage as spectra exhibiting complete peptide fragmentation rarely cover entire target proteins, yet are required to accurately reconstruct full-length peptide sequences.

An alternative approach to separately sequencing individual spectra is to simultaneously interpret multiple MS/MS spectra from overlapping peptides using another process called Shotgun Protein Sequencing (SPS). SPS has been found to generate sequences that frequently cover 90-95% of the target protein sequence(s) while only misidentifying 1 out of every 20 amino acids on a high resolution MS/MS spectra. SPS has limitations. It generates fragmented sequences that do not singularly cover large regions of the target protein sequences, much less complete proteins. SPS sequences have an average length of 10-15 amino acids and the longest recovered SPS de novo sequence is less than 45 amino acids long.

In order to be approved for therapeutic use in humans or animals, biosimilars must be shown to be as close to identical, e.g., have “sameness,” to the parent innovator biologic product based on data compiled through clinical, animal, and analytical studies, as well as conformational status. None of the top-down or bottom-up reversed-phase chromatographic methods provides a reliable and simple basis (e.g., 100% sequence accuracy and coverage) for determining biosimilarity of a recombinant protein.

Therefore, there is a present need for a method for determining analytical similarity or “sameness” of recombinant proteins, e.g., monoclonal antibodies, in comparison to a parent innovator biologic product, wherein the method accurately analyzes amino acid sequence coverage up to 100% with high confidence using a significantly reduced time frame (when compared to well-used protease digestion protocols) for protease digestion of the recombinant protein and enhanced conditions for peptide exposure and consequently increased adherence of peptides to a chromatography column. The method is useful for developing approved biosimilars, as well as quality control analyses during the manufacture of approved biosimilars.

### SUMMARY

The disclosure provides methods for use in evaluating, selecting, and/or manufacturing biologics, including, for example, biosimilars, including interchangeable compositions related thereto (e.g., pharmaceutical preparations). For example, the disclosure provides methods whereby a target protein (e.g., parent innovator biologic product approved under a biologics license application (BLA)) is defined by characteristic signatures, e.g., amino acid sequence, and such signatures are used in the evaluation, identification, and/or manufacture of biologics having the required “sameness” to the target protein for use in diagnostics or approval for use as a therapeutic. The disclosed methods are also useful, for example, for monitoring product changes and controlling product drift that may occur as a result of the use of recombinant technologies with living cells during manufacture of the biologics. The methods include steps for evaluating the similarity of the test protein with a target protein with high reliability on the coverage and accuracy up to 100% of the amino acid sequence of the biologic. For example, the test protein can be evaluated to determine if it has a predetermined level of similarity, or “sameness” with a target protein that is commercially available and/or approved for therapeutic use in humans or animals. This is of particular benefit wherein one or more, or all, of the following conditions is present: (1) the test protein is made by a different method than the target protein or the method used to make the target protein is not known to the maker of the test protein; (2) the test protein is made by an entity having a different marketing approval (or no approval at all) than the entity that makes the target protein; or (3) the test protein was approved in a process that relied on or referred to clinical information regarding the target protein for its approval.

The disclosure provides a method for determining the biosimilarity of a test protein in relation to a target biologic, the method comprising the steps of: (a) digesting a first sample of a test protein for a first incubation time using a first protease and digesting a second sample of the test protein for a second incubation time using a second protease, wherein the first sample and the second sample are physically separated; (b) applying column chromatography and tandem mass spectroscopy to the first sample under conditions sufficient to enhance binding of small peptides to the column, and generating a sequence of the test protein in the first sample; (c) applying column chromatography and tandem mass spectroscopy to the second sample under conditions sufficient to enhance binding of small peptides to the column, and generating the sequence of the test protein in the second sample, wherein the first sample and second sample are physically separated; (d) identifying the test protein as biosimilar to the target biologic when the test protein comprises 100% sequence identity to the target biologic; and (e) identifying the test protein as not biosimilar to the target biologic when the test protein does not comprise 100% sequence identity to the target biologic.

In certain embodiments of method for determining the biosimilarity of a test protein in relation to a target biologic of the disclosure, the monoclonal antibody comprises Adalimumab.

In certain embodiments of method for determining the biosimilarity of a test protein in relation to a target biologic of the disclosure, the first protease is Trypsin. Alternatively, or in addition, in certain embodiments of method for determining the biosimilarity of a test protein in relation to a target biologic of the disclosure, the second protease is Chymotrypsin.

In certain embodiments of method for determining the biosimilarity of a test protein in relation to a target biologic of the disclosure, the first digestion period is about 0.1 to about 1.0 hour. In certain embodiments, the first digestion period is about 0.1 to about 0.5 hour. In certain embodiments, the first digestion period is about 0.6 to about 1.0 hour. In certain embodiments, the first digestion period is about 0.5 hours.

In certain embodiments of method for determining the biosimilarity of a test protein in relation to a target biologic of the disclosure, the second digestion period is about 0.1 to about 2.0 hours. In certain embodiments, the second digestion period is about 0.1 to about 1.5 hours. In certain embodiments, the second digestion period is about 1.5 to about 2.0 hours. In certain embodiments, the second digestion period is about 1.5 hours. The disclosure provides a method for determining the biosimilarity of a test protein in relation to a target biologic, the method comprising the steps of: digesting a first sample of a test protein for a first incubation time using a first protease and a second sample of the test protein for a second incubation time using a second protease, wherein the test protein is digested separately in the first sample and the second sample into peptide sequences; and analyzing the peptide sequences of the first sample separately from the peptide sequences of the second sample using column chromatography to determine 100% of the amino acid sequence coverage and 100% of the amino acid sequence accuracy of the test protein, wherein the column chromatography includes conditions that enhance binding of small peptides to the column.

In certain embodiments of the method for determining the biosimilarity of a test protein in relation to a target biologic, the test protein is one of a protein, a glycoprotein, a fusion protein, a growth factor, a vaccine, a blood factor, a thrombolytic agent, a hematopoietic protein, a hormone, an interferon, an interleukin-based product, an antibody, a monospecific (e.g., monoclonal) antibody, a pegylated antibody, an antibody drug conjugate, a therapeutic enzyme, a cytokine, or a soluble receptor fragment.

In certain embodiments of the method for determining the biosimilarity of a test protein in relation to a target biologic, the first protease is Trypsin. Alternatively, or in addition, in certain embodiments of the method for determining the biosimilarity of a test protein in relation to a target biologic, the second protease is Chymotrypsin.

In certain embodiments of the method for determining the biosimilarity of a test protein in relation to a target biologic, the first protease is Trypsin. In certain embodiments, including those in which the first protease is Trypsin, the first digestion period is about 0.1 to about 1.0 hour. In certain embodiments, including those in which the first protease is Trypsin, the first digestion period is about 0.1 to about 0.5 hour. In certain embodiments, including those in which the first protease is Trypsin, the first digestion period is about 0.6 to about 1.0 hour. In certain embodiments, including those in which the first protease is Trypsin, the first digestion period is about 0.5 hours.

In certain embodiments of the method for determining the biosimilarity of a test protein in relation to a target biologic, the second protease is Chymotrypsin. In certain embodiments, including those in which the second protease is Chymotrypsin, the second digestion period is about 0.1 to about 2.0 hours. In certain embodiments, including those in which the second protease is Chymotrypsin, the second digestion period is about 0.1 to about 1.5 hours. In certain embodiments, including those in which the second protease is Chymotrypsin, the second digestion period is about 1.5 to about 2.0 hours. In certain embodiments, including those in which the second protease is Chymotrypsin, the second digestion period is about 1.5 hours.

In certain embodiments of the method for determining the biosimilarity of a test protein in relation to a target biologic, the target biologic is a commercially available or approved biologic for therapeutic use in humans or animals, a reference listed drug for a secondary approval process, a protein, a glycoprotein, a fusion protein, a growth factor, a vaccine, a blood factor, a thrombolytic agent, a hematopoietic protein, a hormone, an interferon, an interleukin-based product, an antibody, a monospecific (e.g., monoclonal) antibody, a pegylated antibody, an antibody drug conjugate, a therapeutic enzyme, a cytokine, or a soluble receptor fragment. In certain embodiments, the target biologic is one of Adalimumab (Humira®), Bevacizumab (Avastin®), Denosumab (Xgeva®), Cetuximab (Erbitux®); Rituximab (Rituxan®); Mabthera®; Campath®; Herceptin®; Xolair®; Prolia®; Vectibix®; ReoPro®; Zenapax®; Simulect®; Synagis®, Remicade®; Mylotarg®; Campath®; Raptiva®; Zevalin®; Erbitux®; Tysabri®; Lucentis®, Soliris®, Cimzia®; Ilaris®, Arzerra®; Bexxar®; Simponi®; Actemra®; Benlysta®; Adcetris®; or Yervoy®. In certain embodiments of the method for determining the biosimilarity of a test protein in relation to a target biologic, the target biologic is Adalimumab (Humira®).

The disclosure provides a method for analyzing the biosimilarity of a recombinant monoclonal antibody in relation to Adalimumab or its bioequivalent, the method comprising the steps of: determining up to 100% of an amino acid sequence of the recombinant monoclonal antibody by digesting a first sample of the recombinant monoclonal antibody with a first protease and separately digesting a second sample of the recombinant monoclonal antibody with a second protease, wherein the protease digestion steps include incubation times that are no longer than 2 hours collectively; and comparing the amino acid sequence of the recombinant monoclonal antibody to an amino acid sequence of the Adalimumab or its bioequivalent to determine sameness.

In certain embodiments of the method for analyzing the biosimilarity of a recombinant monoclonal antibody in relation to Adalimumab or its bioequivalent, the sameness comprises 100% similarity between the amino acid sequence of the recombinant monoclonal antibody and the amino acid sequence of Adalimumab or its bioequivalent.

The disclosure provides a method for manufacturing a pharmaceutical product comprising a recombinant monoclonal antibody, the method comprising the steps of: providing a recombinant monoclonal antibody, wherein the recombinant monoclonal antibody is not approved under a BLA or a supplemental BLA; acquiring input values for the recombinant monoclonal antibody, wherein one or more of the input values are amino acid sequence(s) of a target biologic; acquiring a plurality of assessments made by comparing the input values with a plurality of amino acid sequence(s) for the target biologic, wherein the target biologic is approved under a biologics license application (BLA) or a supplemental BLA; and processing the recombinant monoclonal antibody into a pharmaceutical product if the input values are indistinguishable from target values for said amino acid sequence(s) for the target biologic.

In certain embodiments of the method for manufacturing a pharmaceutical product comprising a recombinant monoclonal antibody, the recombinant monoclonal antibody is engineered to be a biosimilar to one of Adalimumab (Humira®), Bevacizumab (Avastin®), Denosumab (Xgeva®), Cetuximab (Erbitux®); Rituxan®; Mabthera®; Campath®; Herceptin®; Xolair®; Prolia®; Vectibix®; ReoPro®; Zenapax®; Simulect®; Synagis®; Remicade®; Mylotarg®; Campath®; Raptiva®; Zevalin®; Erbitux®; Tysabri®; Lucentis®, Soliris®, Cimzia®; Ilaris®; Arzerra®; Bexxar®; Simponi®; Actemra®; Benlysta®; Adcetris®; or Yervoy®.

In certain embodiments of the method for manufacturing a pharmaceutical product comprising a recombinant monoclonal antibody, the recombinant monoclonal antibody is engineered to be a biosimilar to Adalimumab (Humira®).

In certain embodiments of the method for manufacturing a pharmaceutical product comprising a recombinant monoclonal antibody, the input values comprise 100% coverage of the amino acid sequence of the recombinant monoclonal antibody.

The disclosure provides a method for analyzing up to 100% of the sequence of amino acids of a recombinant monoclonal antibody to determine sameness to a pharmaceutical product, the method comprising the steps of: fragmenting a denatured recombinant monoclonal antibody into discrete peptides by digesting a first sample of the denatured recombinant monoclonal antibody for a first incubation time using a first protease and a second sample of the denatured recombinant monoclonal antibody for a second incubation time using a second protease, wherein the first incubation time is about 0.1 to about 1.0 hours whereafter the first protease is quenched, and wherein the second incubation time is about 1.0 to about 2.0 hours whereafter the second protease is quenched; analyzing the discrete peptides of the recombinant monoclonal antibody to determine the sequence of amino acids that form the recombinant monoclonal antibody; and comparing the sequence of amino acids of the recombinant monoclonal antibody against a sequence of amino acids of the pharmaceutical product, wherein the pharmaceutical product is approved under a biologics license application (BLA) or a supplemental BLA.

Methods are also provided for the generation of, or evaluation of, a predetermined plurality of target values for the generation of, or evaluation of, a signature, e.g., amino acid sequence, for a test protein, and/or use or application of such information to acquire a sameness/identity value describing the relationship (e.g., structural relationship) between the test protein and the target protein. In some instances, a sameness/identity value can be used to evaluate, identify, and/or produce (e.g., manufacture) a test protein. In some instances, a sameness/identity value is a specification for release of a test protein. Accordingly, disclosed herein are methods useful for evaluating, identifying, and manufacturing an approved biologic.

The method optionally includes a preparation step of separating a test biologic preparation from other isoforms or variants of the test biologic, as well as by-products from manufacturing the same, in a highly purified preparation, e.g., a test protein preparation, wherein the test biologic is not approved under a biologics license application (BLA), a supplemental BLA, or equivalents thereof; and then processing the highly purified test biologic preparation using input values for one or more amino acid sequences for a target biologic.

In one embodiment, the test protein is determined to have an amino acid sequence (e.g., a primary amino acid sequence) that is identical or nearly identical to the target protein amino acid sequence (e.g., 100% match with 0.5% tolerance for sequence variance due to translational errors), and the target protein is approved under a BLA, a supplemental BLA, or equivalents thereof.

In one embodiment, the method comprises the steps of: (1) producing an enriched test protein preparation, wherein the test protein may or may not be approved under a biologics license application (BLA), a supplemental BLA, or equivalents thereof; and (2) processing the test protein preparation to determine that the amino acid sequence is indistinguishable from of the amino acid sequence a target protein, wherein the test protein has an amino acid sequence (e.g., a primary amino acid sequence) that is up to 100% identical to the target protein amino acid sequence, and wherein the target protein is approved under a BLA, a supplemental BLA, or equivalents thereof, thereby manufacturing a pharmaceutical product comprising a protein, e.g., a monoclonal antibody (mAb).

In an embodiment, the target protein is an antibody, e.g., a monoclonal antibody, a humanized antibody, or a human antibody. In alternative embodiments, the target protein can be an antibody conjugated with polyethylene glycol (PEG) polymer chains, e.g., a pegylated antibody. For a pegylated monoclonal antibody, depending on the degree of pegylation and the range of mass size of pegylation, the methods of the disclosure can be adopted to sequence the amino acids of the monoclonal antibody after a step of releasing PEG prior to sample preparation for peptide mapping. In further embodiments, the target protein can be an antibody-drug conjugate (ADC) complex molecule composed of an antibody, e.g., whole mAb or an antibody fragment such as a single-chain variable fragment (scFv)) that is linked, via a stable, chemical linker with labile bonds, to a biological active cytotoxic (anticancer) payload or drug. In an ADC complex molecule, wherein the reactive residue with the drug is modified, the methods of the disclosure can be used to map the drug conjugation sites by including the molecular weight of the drug as a modification in the sequence database.

For example, the target protein can be selected from the products marketed as Adalimumab (Humira®), Bevacizumab (Avastin®), Denosumab (Xgeva®), Cetuximab (Erbitux®); Rituxan®; Mabthera®; Campath®; Herceptin®; Xolair®; Prolia®; Vectibix®; ReoPro®; Zenapax®; Simulect®; Synagis®; Remicade®; Mylotarg®; Campath®; Raptiva®; Zevalin®; Erbitux®; Tysabri®; Lucentis®; Soliris®; Cimzia®; Ilaris®; Arzerra®; Bexxar®; Simponi®; Actemra®; Actemra®; Benlysta®; Adcetris®; or Yervoy®, as well as other biologics.

### BRIEF DESCRIPTION OF THE DRAWINGS

Additional aspects, features, and advantages of the disclosure, both as to its methods and use, will be understood and become more readily apparent when the disclosure is considered in light of the following description of illustrative embodiments made in conjunction with the accompanying drawings, wherein:

FIGS. 1A-B are a pair of graphs illustrating chromatographic profiles of trypsin-digested and chymotrypsin-digested chromatography matrix, respectively, that were run for specificity. The matrix showed no hit on any target amino acid sequence on Sequence Discoverer. Small peaks on the matrix chromatograms show system peaks and enzyme peaks.

FIGS. 2A-D are a series of alignments illustrating sequence coverage for trypsin-digested heavy chain (FIG. 2A (SEQ ID NO: 1, with potential modifications (SEQ ID NO: 2)), chymotrypsin-digested heavy chain (FIG. 2B (SEQ ID NO: 3, with potential modifications (SEQ ID NO: 4)), trypsin-digested light chain (FIG. 2C (SEQ ID NO: 5, with potential modifications (SEQ ID NO: 6)), and chymotrypsin-digested light chain (FIG. 2D (SEQ ID NO: 7, with potential modifications (SEQ ID NO: 8)) of the ONS-3010 reference standard (Adalimumab), respectively. These figures demonstrate that the method of the disclosure is capable of 100% sequence coverage.

FIGS. 3A-B are a pair of graphs illustrating chromatographic profiles for trypsin-digested and chymotrypsin-digested ONS-3010 reference standards, respectively, that were run for specificity.

FIGS. 4A-D are a series of alignments illustrating 100% sequence coverage for trypsin-digested heavy chain (FIG. 4A (SEQ ID NO: 9, with potential modifications (SEQ ID NO: 10)), chymotrypsin-digested heavy chain (FIG. 4B (SEQ ID NO: 11, with potential modifications (SEQ ID NO: 12)), trypsin-digested light chain (FIG. 4C (SEQ ID NO: 13, with potential modifications (SEQ ID NO: 14)), and chymotrypsin-digested light chain (FIG. 4D (SEQ ID NO: 15, with potential modifications (SEQ ID NO: 16)) of the positive control Adalimumab (Humira®) (sample test ID H35), respectively. These figures confirm that the target sequence is an accurate amino acid sequence for Adalimumab (Humira®).

FIGS. 5A-B are a pair of graphs illustrating chromatographic profiles for trypsin-digested and chymotrypsin-digested positive control Adalimumab (Humira®) (sample test ID H35), respectively.

FIGS. 6A-D are a series of alignments illustrating sequence coverage for trypsin-digested heavy chain (FIG. 6A (SEQ ID NO: 17, with potential modifications (SEQ ID NO: 18)), chymotrypsin-digested heavy chain (FIG. 6B (SEQ ID NO: 19, with potential modifications (SEQ ID NO: 20)), trypsin-digested light chain (FIG. 6C (SEQ ID NO: 21, with potential modifications (SEQ ID NO: 22)), and chymotrypsin-digested light chain (FIG. 6D (SEQ ID NO: 23, with potential modifications (SEQ ID NO: 24)) of the negative control Rituximab (Rituxan®) (sample test ID M6), respectively. This demonstrates that the method of the disclosure is capable of identifying sequences accurately.

FIGS. 7A-B are a pair of graphs illustrating chromatographic profiles of trypsin-digested and chymotrypsin-digested negative control Rituximab (Rituxan®) (sample test ID M6), respectively.

FIG. 8 is a schematic diagram illustrating the theoretical amino acid sequences of the light chain ((SEQ ID NO: 25) and the heavy chain of Adalimumab (Humira®) ((SEQ ID NO: 26).

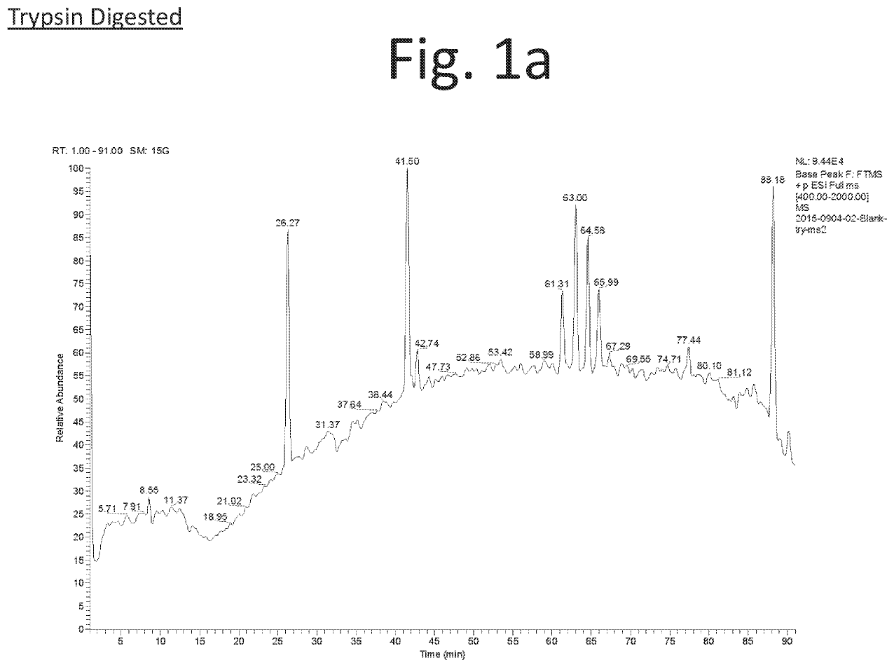

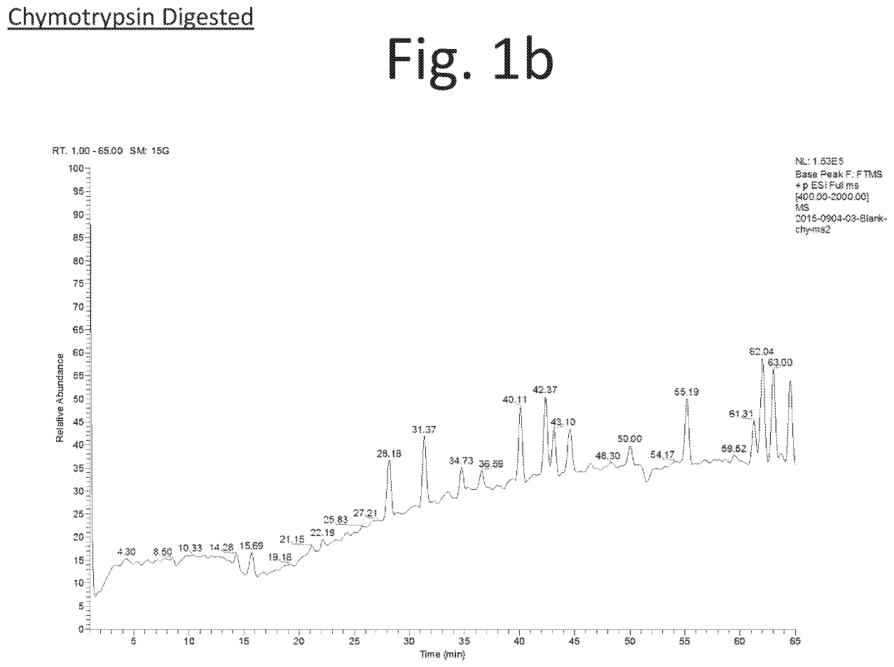

### DETAILED DESCRIPTION

As used herein, the term “biologic” (singular or plural) refers to peptide and protein products. For example, biologics include naturally-derived or recombinant products expressed in cells, such as, e.g., proteins, glycoproteins, fusion proteins, growth factors, vaccines, blood factors, thrombolytic agents, hormones, interferons, interleukin-based products, monospecific (e.g., monoclonal) antibodies, therapeutic enzymes. Biologics may be approved under a biologics license application (BLA), under Section 351(a) of the Public Health Service (PHS) Act, whereas biosimilar and interchangeable biologics referencing a BLA as a reference product are licensed under Section 351(k) of the PHS Act. Section 351 of the Public Health Service (PHS) Act is codified as 42 U.S.C. 262. Other biologics may be approved under Section 505(b)(1) of the Federal Food and Cosmetic Act, or as abbreviated applications under Sections 505(b)(2) and 505(j) of the Hatch Waxman Act, wherein Section 505 is codified as 21 U.S.C. 355.

As used herein, the term “isoform” (singular or plural) refers to any of several different forms of the same protein, arising from either single nucleotide polymorphisms, differential splicing of mRNA, or post-translational modifications (e.g., sulfation, glycosylation, etc.).

As used herein, the term “antibody” (singular or plural) refers, in the broadest sense, to monoclonal antibodies (including full length monoclonal antibodies) of any of the classes IgG, IgM, IgD, IgA, and IgE, as well as antibody fragments that exhibit a desired biological activity. The phrase “antibody fragments” refers to a portion of a full-length antibody, generally the antigen binding or variable region thereof. Examples of antibody fragments include Fab, Fab′, F(ab′)2, and Fv fragments; diabodies; linear antibodies; single-chain antibody molecules; and multi-specific antibodies formed from antibody fragments.

As used herein, the term “monoclonal antibody” (singular or plural) refers to antibodies that are highly specific, being directed against a single antigenic epitope. Alternatively, the term “monoclonal antibody” refers to an antibody produced from a single spleen cell clone. In a non-limiting example, a monoclonal antibody can be a fully humanized antibody, i.e., both its variable and constant region are derived from a human source.

As used herein, the term “approval” refers to the procedure by which a regulatory entity, e.g., the USFDA, approves a candidate for therapeutic or diagnostic use in humans or animals. As used herein, a primary approval process is an approval process which does not refer to a previously approved protein, e.g., it does not require that the protein being approved have structural or functional similarity to a previously approved protein, e.g., a previously approved protein having the same primary amino acid sequence or a primary amino acid sequence. In embodiments, the primary approval process is one in which the applicant does not rely, for approval, on data, e.g., clinical data, from a previously approved product. Exemplary primary approval processes include, in the United States, a Biologics License Application (BLA), or supplemental Biologics License Application (sBLA), a new drug application (NDA) under Section 505(b)(1) of the Federal Food and Cosmetic Act, and, in Europe, an approval in accordance with the provisions of Article 8(3) of the European Directive 2001/83/EC, or an analogous proceeding in other countries or jurisdictions.

As used herein, the term “glycoprotein” refers to an amino acid sequence that includes one or more oligosaccharide chains (e.g., glycans) covalently attached thereto. Exemplary amino acid sequences include peptides, polypeptides, and proteins. Exemplary glycoproteins include glycosylated antibodies and antibody-like molecules (e.g., Fc fusion proteins). Exemplary antibodies include monoclonal antibodies and/or fragments thereof, polyclonal antibodies and/or fragments thereof, and Fc domain containing fusion proteins (e.g., fusion proteins containing the Fc region of IgG1, or a glycosylated portion thereof). A glycoprotein preparation is a composition or mixture that includes at least one glycoprotein.

As used herein, the phrase “target biologic”, e.g., target protein, refers to a commercially available, or approved, biologic which defines or provides the basis against which a test biologic is measured or evaluated. In embodiments, a target biologic is commercially available for therapeutic use in humans or animals. In other embodiments, the target biologic is approved for use in humans or animals by a primary approval process. In further embodiments, the target biologic is a reference listed drug for a secondary approval process. An exemplary target protein is an antibody, e.g., humanized or human antibody. Other target proteins include glycoproteins, cytokines, hematopoietic proteins, soluble receptor fragments, and growth factors.

As used herein, the term “evaluating” refers to reviewing, considering, determining, assessing, measuring, and/or detecting the presence, absence, level, and/or ratio of one or more parameters in a test protein and/or target biologic to provide information pertaining to the one or more parameters. In some instances, evaluating a glycoprotein preparation includes detecting the presence, absence, level, or ratio of one or more points of similarity between a test protein and a target biologic.

As used herein, the term “analyzing” refers to performing a process that involves a physical change in a sample or another substance, e.g., a starting material. Exemplary changes include making a physical entity from two or more starting materials, shearing or fragmenting a substance, separating or purifying a substance, combining two or more separate entities into a mixture, or performing a chemical reaction that includes breaking or forming a covalent or non-covalent bond. Analyzing a sample can include performing an analytical process which includes a physical change in a substance, e.g., sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

As used herein, the phrase “input value” refers to a value associated with a parameter of a test biologic. The value can be qualitative, e.g., present, absent, intermediate, or the value can be qualitative, e.g., it can be a numerical value such as a single number, or a range, for a parameter.

### General Method of the Disclosure

The methods of the disclosure can be used for analytically determining similarity of a recombinant protein (e.g., test protein) to a parent innovator biologic product (e.g., target protein) throughout the development and manufacture of biosimilar therapeutic molecules.

Non-limiting applications of the method include use in determining the similarity of recombinant proteins to biologic products including, but not limited to, Adalimumab (Humira®), Bevacizumab (Avastin®), Denosumab (Xgeva®), Cetuximab (Erbitux®); Rituximab (Rituxan®); Mabthera®; Campath®; Herceptin®; Xolair®; Prolia®; Vectibix®; ReoPro®; Zenapax®; Simulect®; Synagis®; Remicade®; Mylotarg®; Campath®; Raptiva®; Zevalin®; Erbitux®; Tysabri®; Lucentis®; Soliris®; Cimzia®; Ilaris®; Arzerra®; Bexxar®; Simponi®; Actemra®; Benlysta®; Adcetris®; and Yervoy®, as well as other biologics.

The method provides an analysis for evaluating the primary structure of a test protein, either for analyzing a test protein or a target protein, and/or for analyzing the test protein in comparison to a target protein. This method provides up to 100% amino acid sequence coverage and accuracy.

In an embodiment, a test protein, such as a recombinant protein that can include variants, can be analyzed. In a non-limiting alternative embodiment, a test protein can optionally be initially purified by column chromatography, e.g., HPLC, to separate the biologic from basic and acidic variants of the biologic and/or any other by-products of manufacture of the biologic, e.g., enzymes, cells, and cellular debris, etc. The biologic, its variants, and related manufacturing by-products can be processed through a chromatographic system, e.g., cation-exchange column, that is capable of high-efficiency, high resolution separation of closely eluting proteins.

In a further non-limiting example, the disclosure provides methods for identifying and confirming the primary structure of the test protein—a monoclonal antibody (e.g., ONS-3010 a biosimilar to the monoclonal antibody Adalimumab (Humira®))—for characterization.

In accordance with the general methods of the disclosure, the test protein and/or target protein, e.g., monoclonal antibody (mAb), is denatured, reduced, alkylated, and spun down through a 10 kDa centrifuge filter. This involves optimal digestion and complete sequence coverage by solubilization of the test protein, denaturation of the test protein, and disulphide bond reduction. Discrete peptides are selectively fragmented by using trypsin (Try) and chymotrypsin (Chy). This peptide mixture is injected onto a reverse-phase ultra-high performance liquid chromatography (RP-UPLC) system to obtain a unique profile (peptide map). The exact mass charge ratios (m/z) of the peptides are determined by full scan on a high resolution mass spectrometer. The peptide is then broken into ion fragments for MS/MS amino acid sequence analysis. The MS/MS data can be analyzed by using, for example, Proteome Discoverer software against an Adalimumab amino acid sequence database to identify peptide sequence. The similarity of amino acid sequences between reference product or target product, and test product is reported.

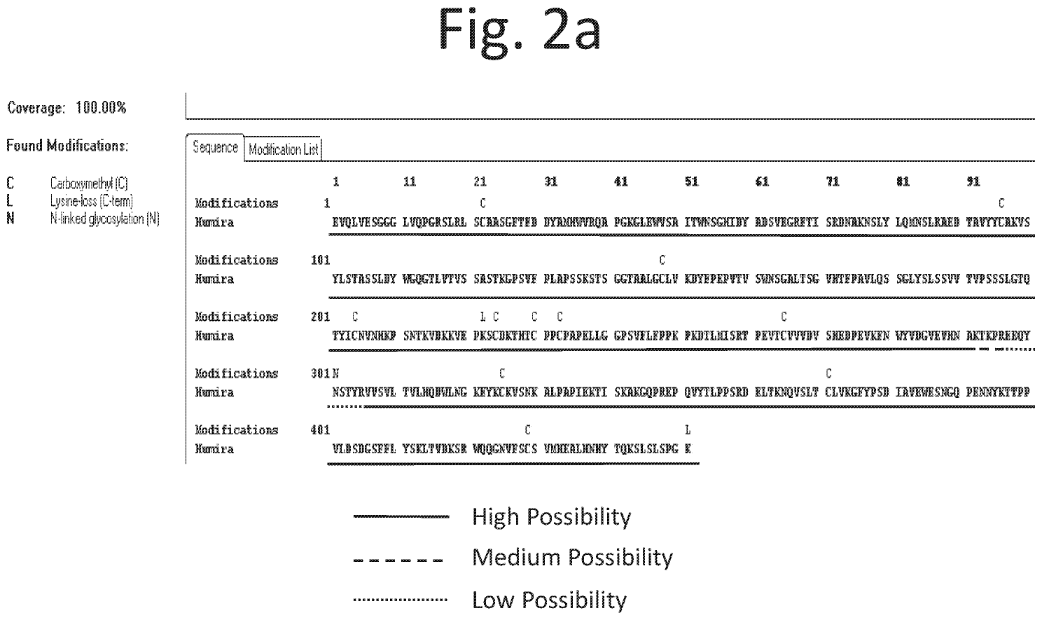

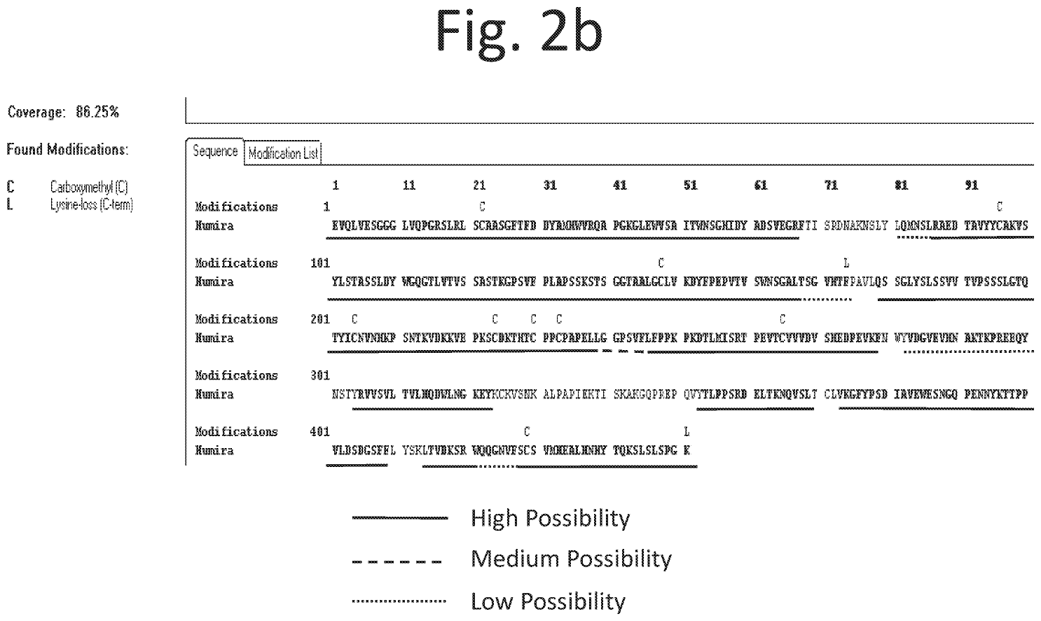

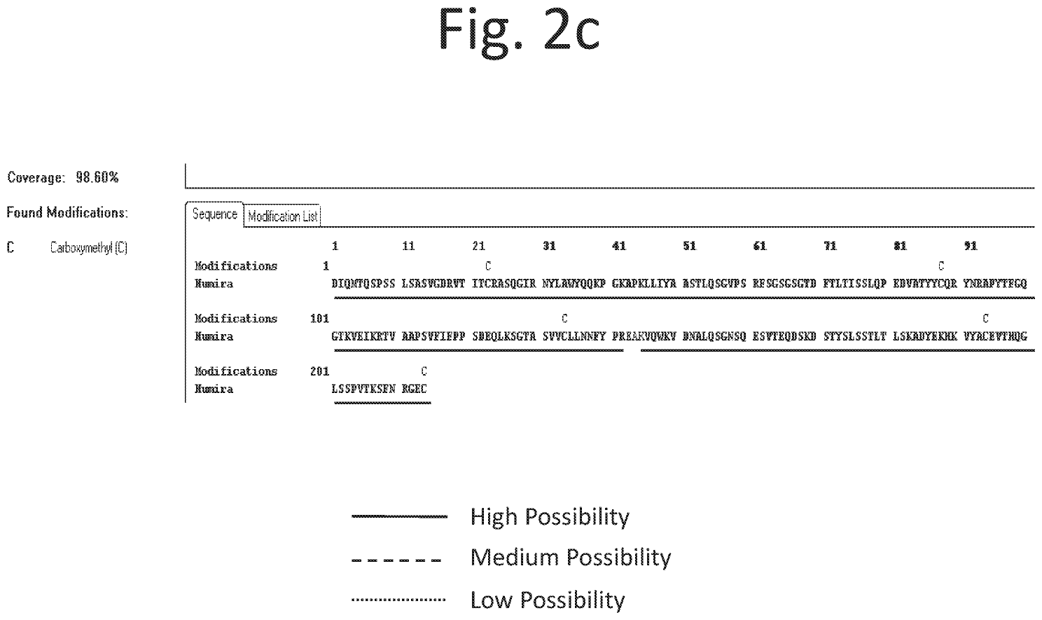

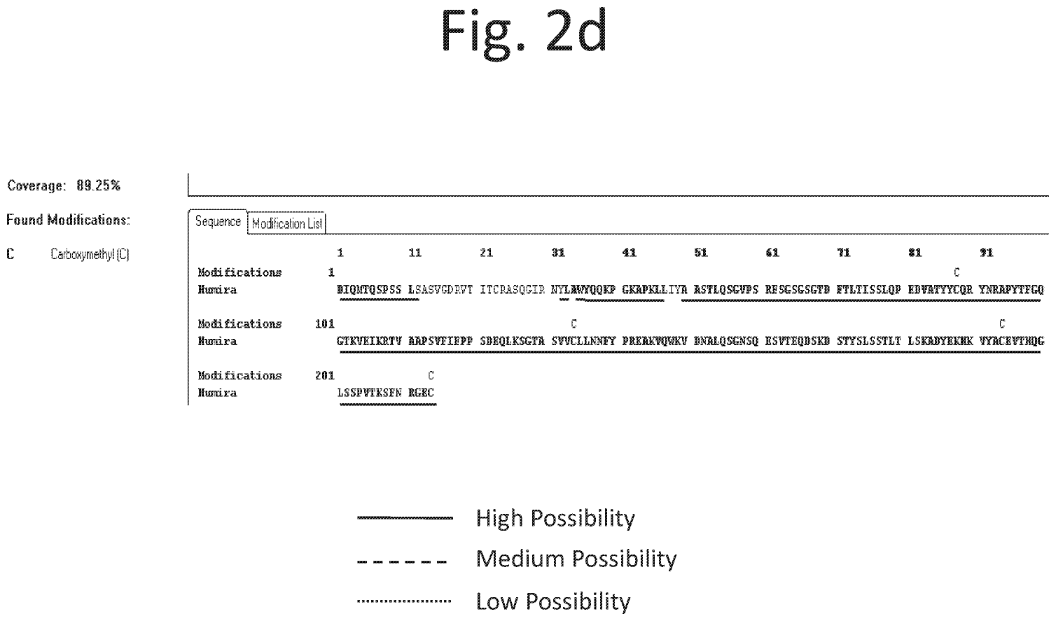

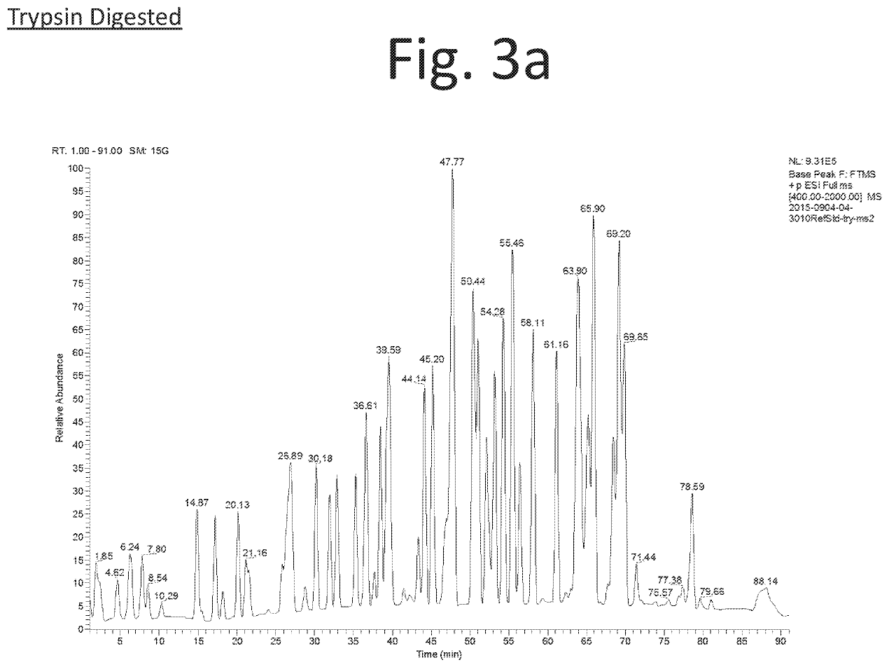

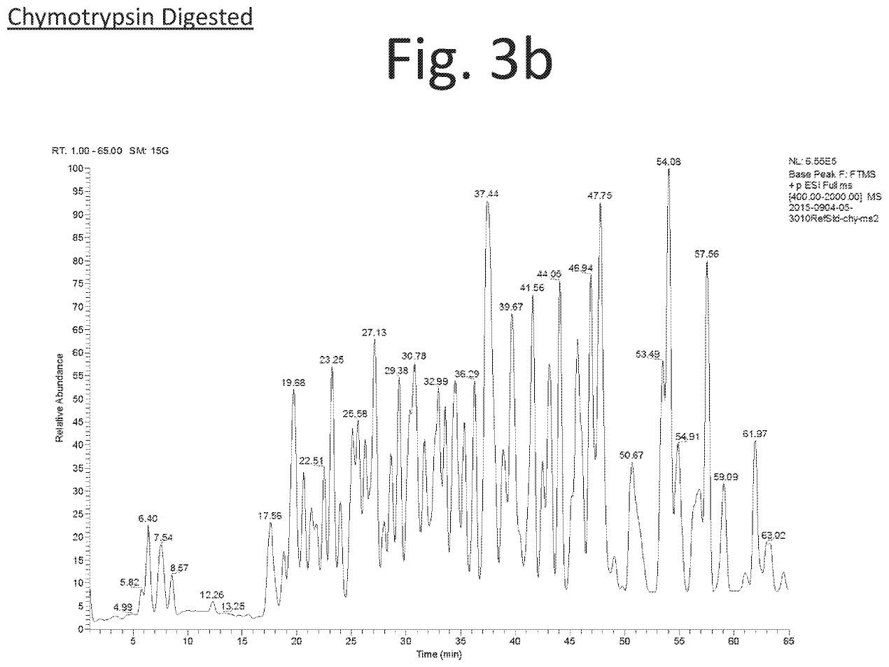

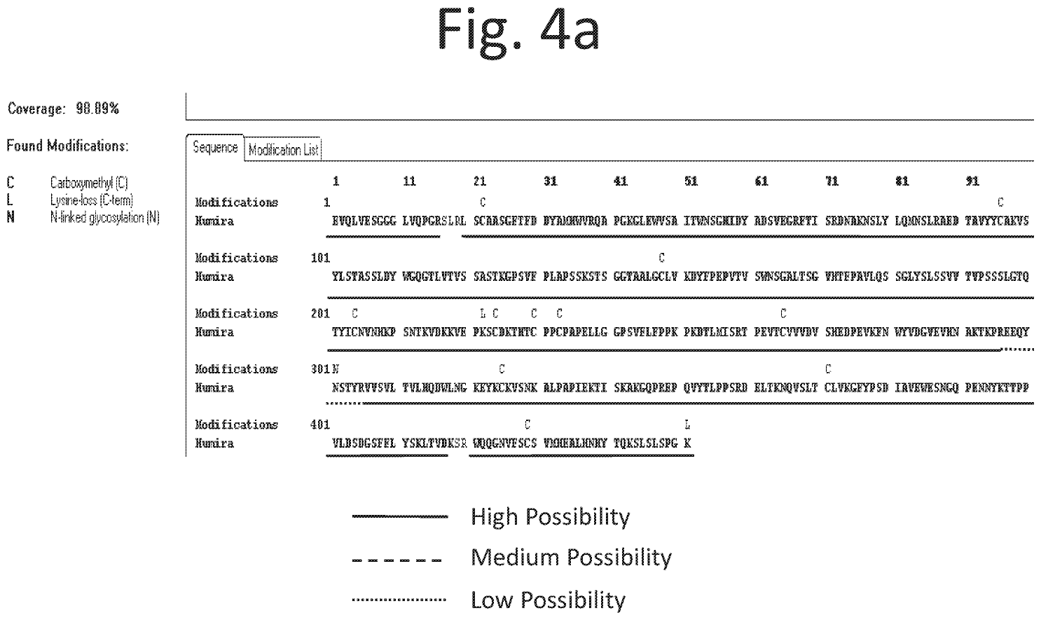

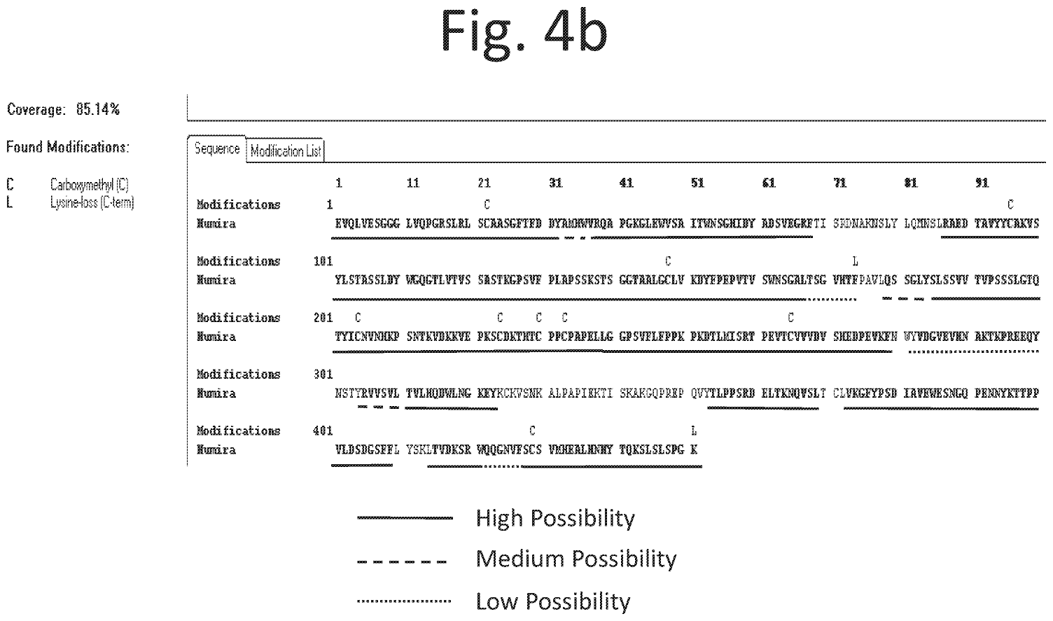

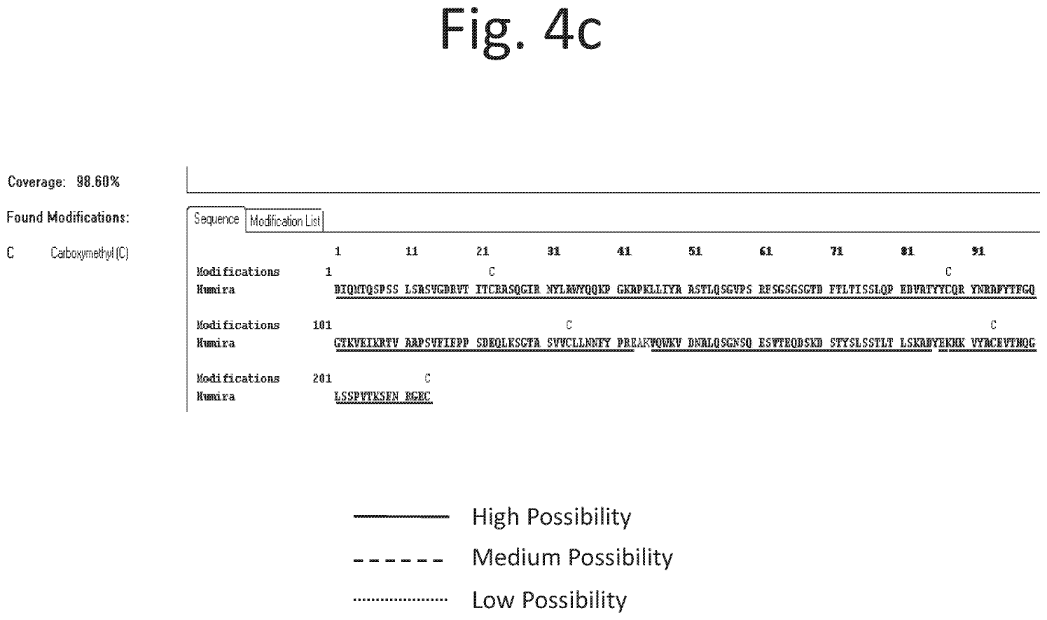

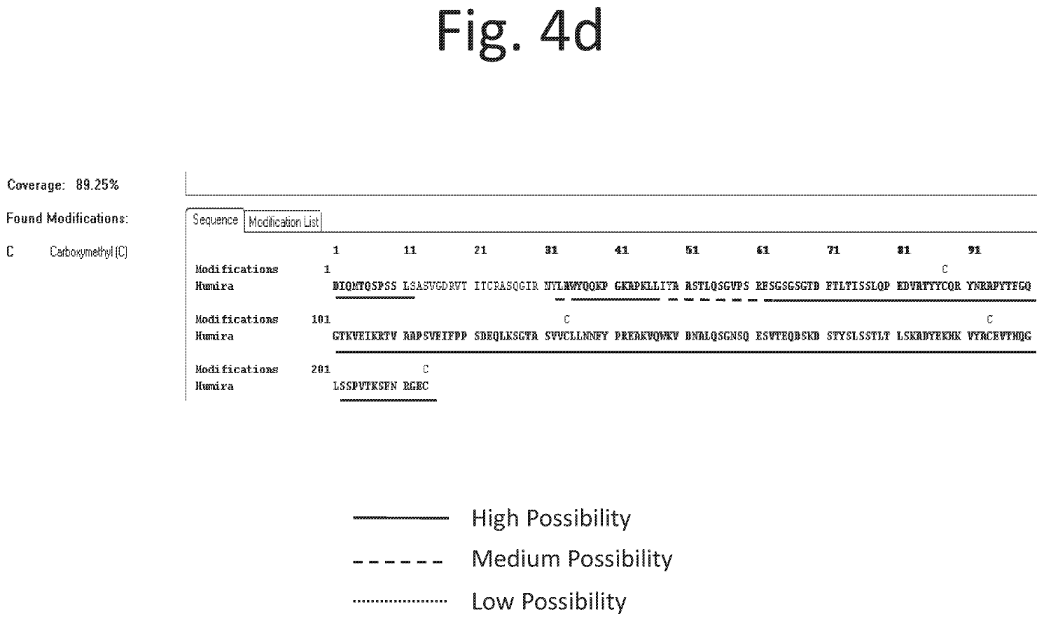

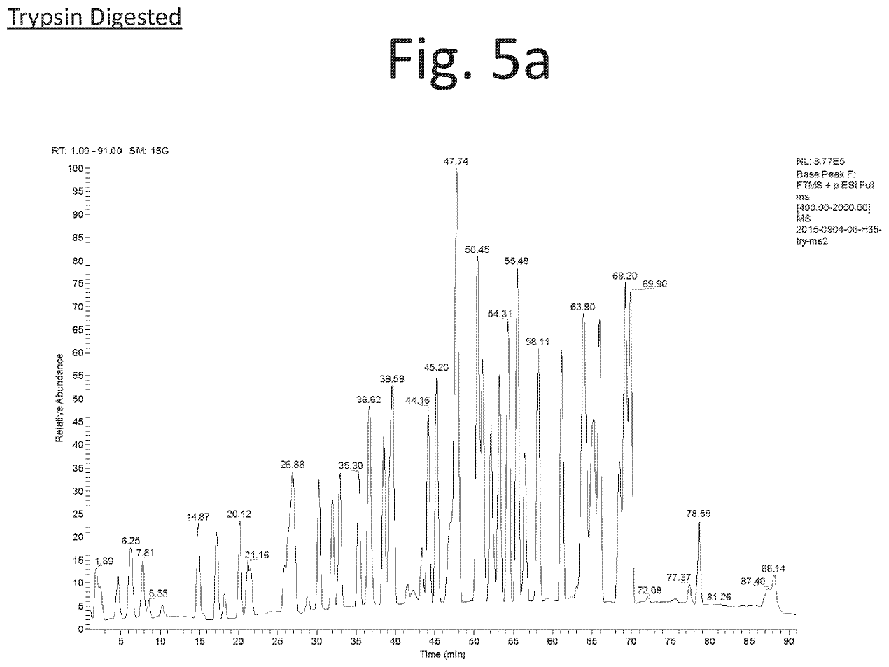

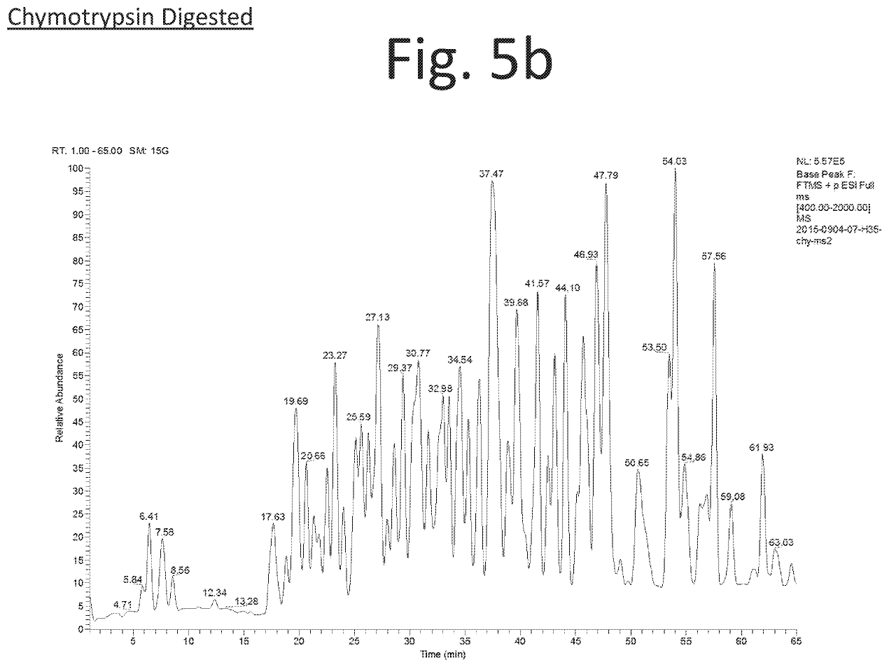

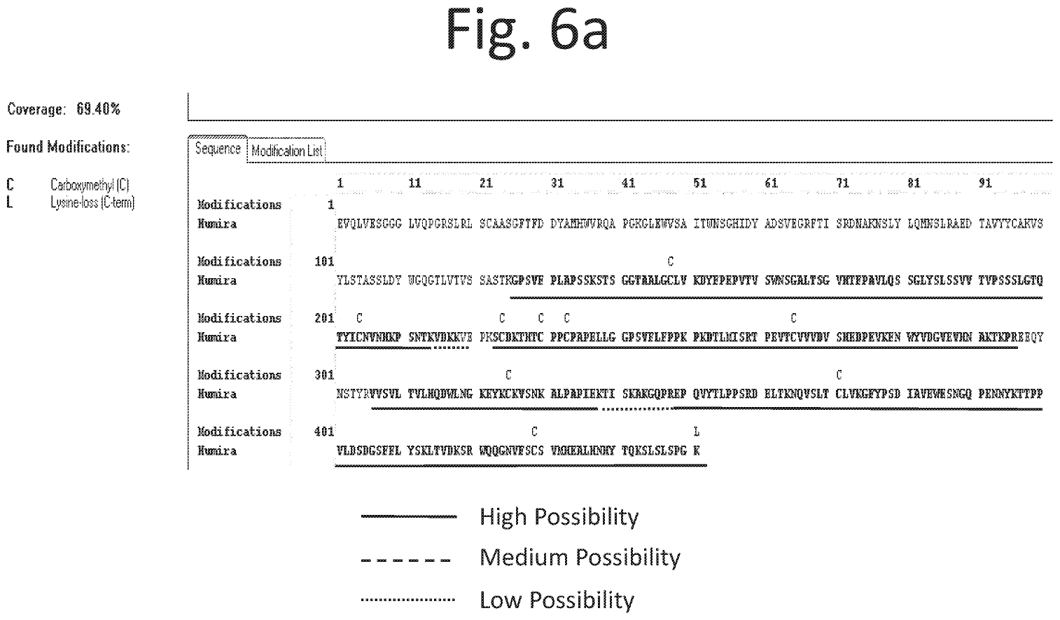

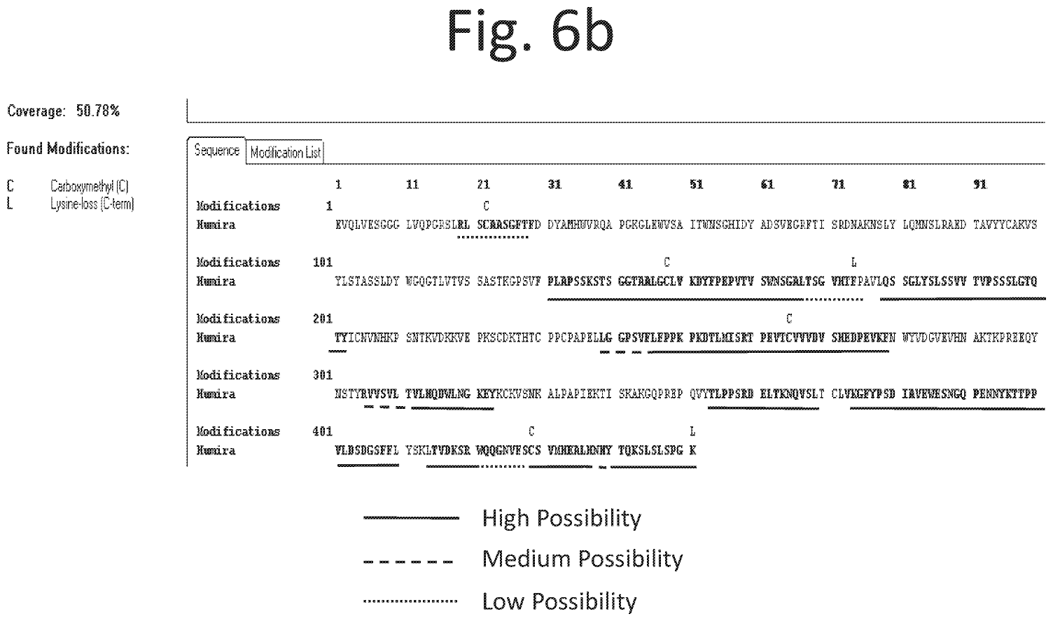

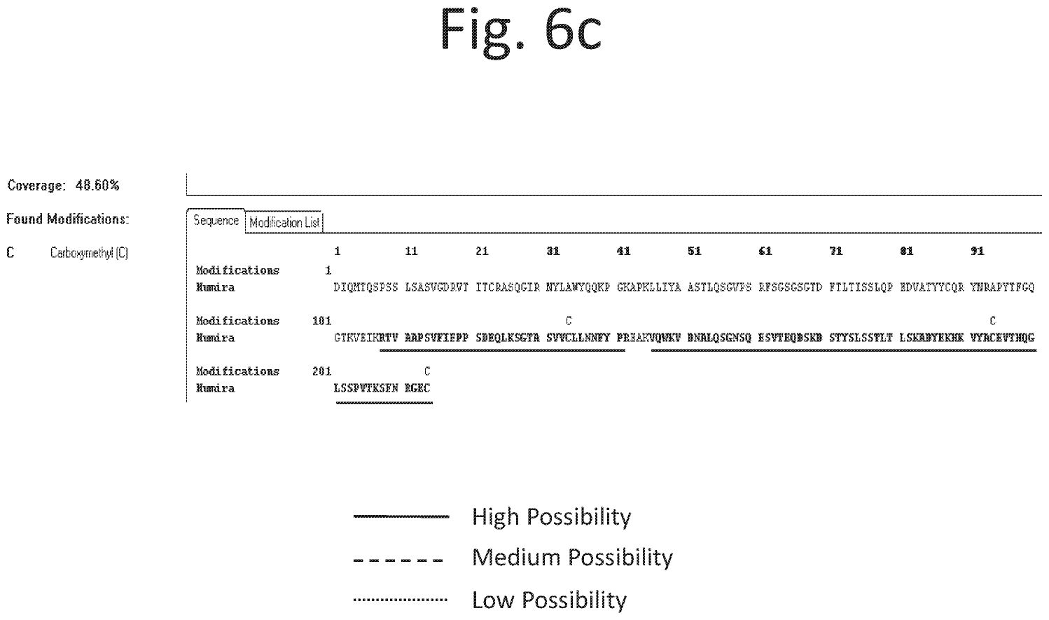

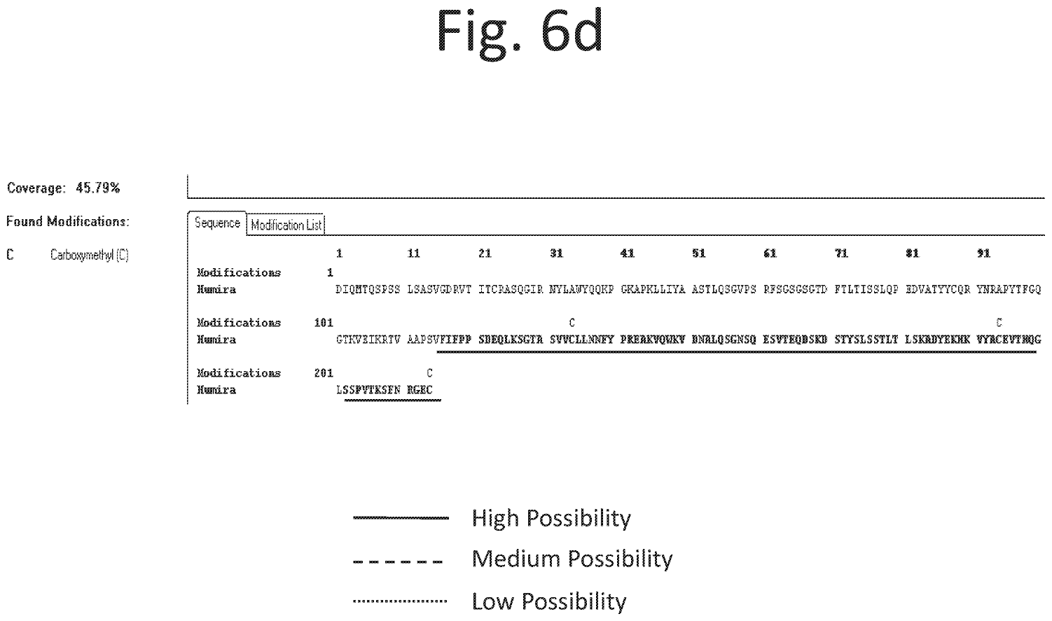

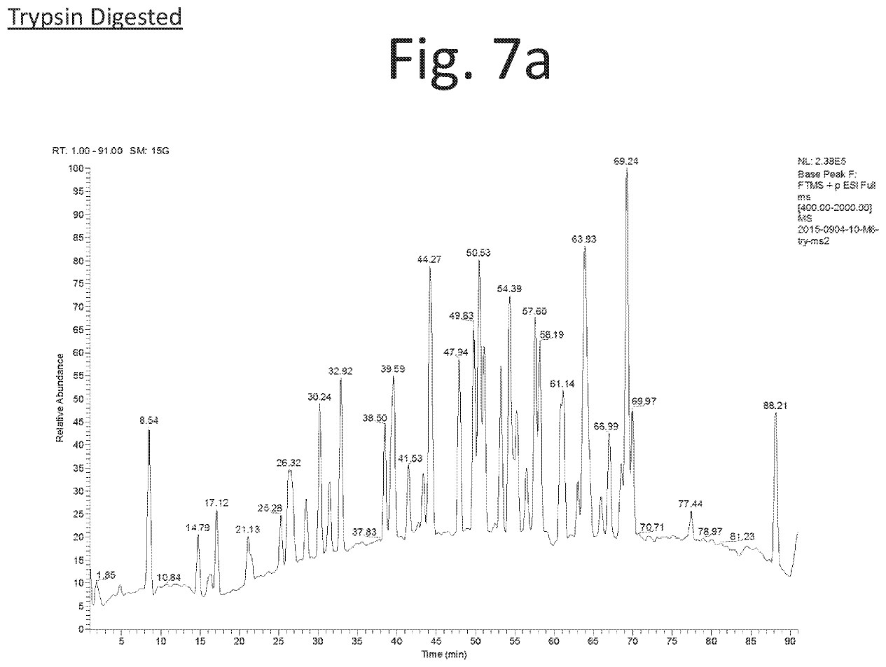

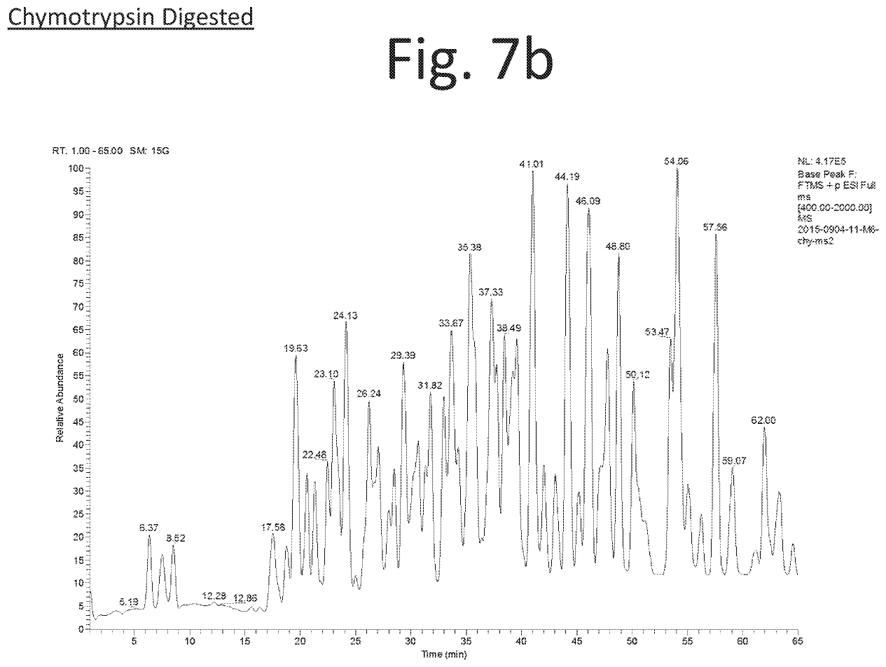

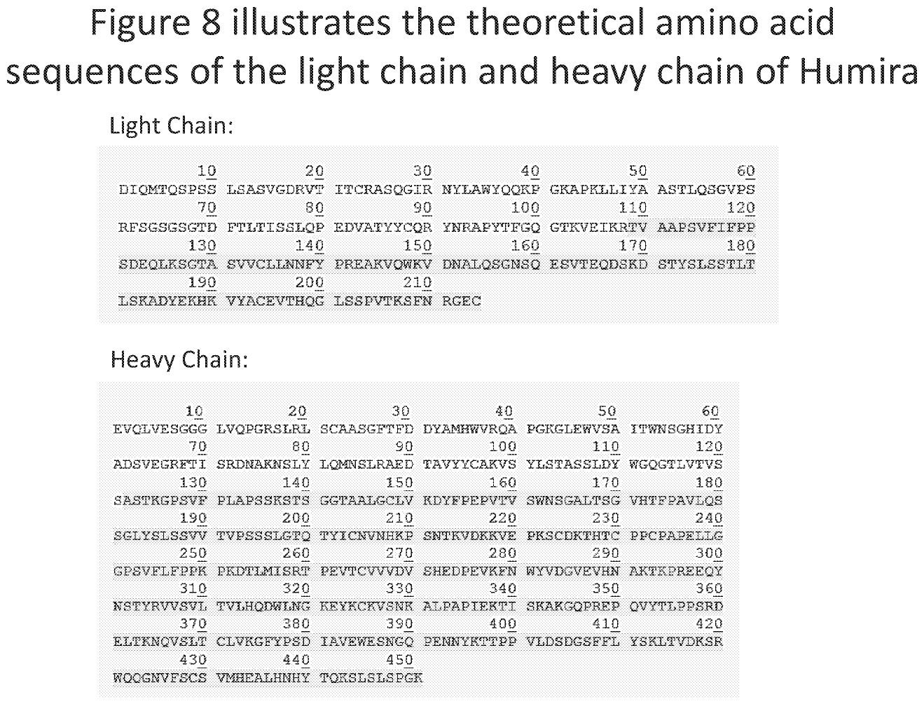

With its application of identifying sequences, the Proteome Discoverer software extracts relevant MS/MS spectra from the “.raw” file and determines the precursor charge state and the quality of the fragmentation spectrum. The SEQUEST search algorithm correlates experimental MS/MS spectra through comparisons to theoretical MS/MS spectra from protein databases. The Proteome Discoverer uses a probability-based scoring system to rate the relevance of the best matches found by the SEQUEST algorithm. The algorithm color codes the amino acid table to show the portion of the corresponding peptide sequence that is identified. Green, yellow, and pink indicate high, medium, and low confidence, respectively. No color means no hit on the peptide. The Protein Results View highlights the fragment ions in a peptide MS/MS spectrum that match predicted fragment masses. Specifically in FIGS. 2A-2D, 4A-4D, and 6A-6D of this disclosure, high, medium and low confidence hits are indicated by a solid line ( [symbol] ), a long broken line ( [symbol] ), and a short broken line (......) instead of color, respectively.

In a specific example, two separate aliquots of the monoclonal antibody (target protein, e.g., Adalimumab and/or bioequivalents) are prepared at a concentration of ≥3.0 mg/mL by transferring water into a 1.5 mL polypropylene centrifuge tube and adding 300 μg of sample into the tube. Negative controls (e.g., formulation buffer or HPLC-grade water) and positive controls (e.g., reference standard) are also prepared as a reference. The peptide standard mixture used as an instrument system suitability control is a 20 μg/mL HPLC peptide standard mixture prepared by adding 2.5 mL of Mobile Phase A (see below) to one vial of standard mixture (Sigma, Cat #H2016-1VL).

Each aliquot of monoclonal antibodies is denatured by adding a mixture of 500 μL of 8N Guanidine HCl (Fisher, Cat #24115), 40 μL of 2.5 M Tris base (3.03 g of Tris base in HPLC water to a final volume of 10 mL), and 20 μL of 1N HCl (Fisher Scientific, Cat #SA48-1 or equivalent) into each tube.

A stabilizing reagent, e.g., Dithiothreitol (DTT), is added to each sample of the denatured protein under conditions that promote the disruption of disulfide bonds of the denatured protein. Each sample of the denatured monoclonal antibodies is then reduced by adding 20 μL of 25 mg/mL of Dithiothreitol (DTT) (Bio-Rad, Cat #161-0611) (e.g., 25 mg of DTT in HPLC Grade water (Fisher Scientific, Cat #W5-4) to a final volume of 1.0 mL) to each sample. The samples are incubated separately at about 37±2° C. for about 0.5 hour.

At the end of the incubation, each sample undergoes alkylation by adding 8 μL of 200 mg/mL sodium iodoacetate (Sigma, Cat #12512) (e.g., 200 mg of sodium iodoacetate mixed with HPLC water to a final volume of 1 mL) to each sample, and then the samples are incubated in the dark at ambient temperature for about 15 minutes.

At the end of the alkylation incubation, each sample is desalted. The desalting process involves washing each sample in a Millipore Biomax-10 kDa Ultrafree 0.5 Centrifuge filter. The filter is initially wetted by centrifuging about 300 μL ammonium bicarbonate centrifuged at 10,000 rpm for 5 minutes. Each sample is transferred to the surface of a pre-wetted filter and is then washed with 300 μL of 0.1 M ammonium bicarbonate and centrifuged at 10,000 rpm for 10 minutes. The wash step can be repeated up to 2 or more times. The final wash involves centrifugation for about 10-13 minutes at 10,000 rpm so that each sample has a final volume of about 100 μL.

The desalted samples are then enzymatically digested by adding a different protease, e.g., trypsin and chymotrypsin, to the samples at optimized incubation conditions that include a reduced time frame for digestion, e.g., up to 0.5 hour for trypsin and up to 1.5 hours for chymotrypsin. The reduced incubation times are shorter in duration than traditional incubations times, e.g., 2-4 hours and even up to 18 hours. The shorter digestion time period provides more instances of specific miscleavage of amino acids so that the glycoprotein produced by digestion comprises longer peptides, rather than shorter peptides produced by longer digestion time. The digestion occurs in two desalted samples individually, namely, one sample is digested using trypsin that is quenched after passage of a first incubation time period, e.g., 0.5 hour, and then a second sample is digested using chymotrypsin that is quenched after passage of a second incubation time, e.g., 1.5 hours. This use of chymotrypsin protease digestion supports the coverage for a small peptide EAK in light chain and a small peptide SLR in heavy chain to archive 100% sequence coverage. During the digestion, the polypeptide chain of the denatured protein is cut into shorter fragments as the enzymes split peptide bonds that link amino acid residues in the denatured protein.

The first sample undergoes proteolytic digestion with trypsin. For example, trypsin (sequence grade modified, Promega, Cat #V5111, 20 μg) can be reconstituted in 20 μL of reconstitution buffer. 15 μL of reconstituted trypsin is added to each sample to reach a ratio of trypsin-to-sample ratio of about 1:20. The sample with trypsin added is incubated at 37° C. for 0.5 hour and then about 5 μL of 10% Formic acid (v/v) (Thermo, Product #28905 or equivalent, e.g., 100 μl of formic acid in 900 μl of HPLC grade water) is added to each sample to quench the enzymatic digestion in preparation for the second digestion.

The second sample undergoes proteolytic digestion with chymotrypsin. For example, chymotrypsin (Promega Chymotrypsin Sequencing Grade, 25 μg, Cat #V1062) can be reconstituted into 20 μL of 1 mM of HCl in HPLC-grade water (Fisher Scientific, Cat #SA48-1 or equivalent) reconstitution buffer. About 15 μL of reconstituted chymotrypsin is added to each sample at a chymotrypsin-to-sample ratio of about 1:16. The sample with chymotrypsin added is incubated at 37° C. for 1.5 hours and then about 5 μL of 10% Formic acid (v/v) is added to each sample to quench the enzymatic digestion.

The samples of digested monoclonal antibodies are then run separately through UPLC-MS/MS for analysis under conditions that promote adsorption of the peptides to the column including smaller chain peptides that are less likely to be bound under normal conditions. For example, the UPLC column can be an UPLC column (Waters BEH300 C18, 2.1×100 mm, 1.7 μm, Cat #186003555) having a pre-column (VanGuard, BEH300 C18, 2.1×5 mm, 1.7 μm, Cat #186003975). Samples are injected into the column at a volume of about 10 μL having a protein concentration of about 3 μg/μL at a temperature of about 45° C. After the sample is loaded on the column, a gradient consisting of Mobile Phase A (0.1% TFA in water (Optima LC/MS, Fisher Scientific, Cat #LS119)) and Mobile Phase B (0.085% TFA in 95% Acetonitrile (ACN) (v/v)) (HPLC grade, Fisher Scientific, Cat #A-998-4 or equivalent), and are passed through the column at a flow rate of about 400 μL/min. The UPLC system parameters are as follows: the autosampler is set at about 4° C.; data rate 20 pts/sec; PMT gain at 1; and PDA wavelength 210 nm and 280 nm.

The gradient for the peptide standard mixture is run as follows:

| Time (min) | % Mobile Phase A | % Mobile Phase B |
|---|---|---|
| 0 | 100 | 0 |
| 7 | 67 | 33 |
| 7.5 | 59 | 41 |
| 8 | 30 | 70 |
| 8.5 | 30 | 70 |
| 9 | 100 | 0 |
| 10 | 100 | 0 |

The gradient for a sample is run as follows:

| Time (min) | % Mobile Phase A | % Mobile Phase B |
|---|---|---|
| 0 | 100 | 0 |
| 10 | 100 | 0 |
| 15 | 98 | 2 |
| 75 | 67 | 33 |
| 85 | 60 | 40 |
| 90 | 30 | 70 |
| 95 | 30 | 70 |
| 96 | 100 | 0 |
| 98 | 100 | 0 |

The sample batch may be set as follows:

| Description | Method | # Injections |
|---|---|---|
| Equilibration Run | Equilibration | 1 |
| Water | Peptide std. | 2 |
| Peptide Standard | Peptide std. | 2 |
| Water | Peptide std. | 2 |
| Trypsin-Buffer Blank | Peptide 98 min run-MS/MS | 1 |
| Water | Peptide std. | 2 |
| Trypsin-ONS3010- | Peptide 98 min run-MS/MS | 1 |
| Ref Std. | | |
| Water | Peptide std. | 2 |
| Trypsin-ONS2010- | Peptide 98 min run-MS/MS | 1 |
| Sample | | |
| Water | Peptide std. | 2 |
| Chymotrypsin-Buffer | Peptide 98 min run-MS/MS | 1 |
| Blank | | |
| Water | Peptide std. | 2 |
| Chymotrypsin- | Peptide 98 min run-MS/MS | 1 |
| ONS3010-Ref Std. | | |
| Water | Peptide std. | 2 |
| Chymotrypsin- | Peptide 98 min run-MS/MS | 1 |
| ONS3010- Sample | | |
| Water | Peptide std. | 2 |
| Peptide Standard | Peptide std. | 1 |
| Water | Peptide std. | 2 |
| Water | Shutdown | 1 |

Amino Acid Sequence Coverage Search Proteome Discoverer 1.3 was used for data analysis of each UPLC run.

Data acceptance, record, and report included exact mass calibration followed by application of the peptide standard layout of Xcalibur onto peptide standard injections. Record 5 peaks of RT of peptide standard injection on MS chromatograms, calculate % RSD of each peak RT individually. Record exact mass of selected peptide at m/z 532 and calculate the mass accuracy for all injections.

The peptide standard layout was as follows:

$$Mass Accuracy ( ppm ) = \frac{\vert Exact Mass - Theoretical Mass \vert}{Exact Mass} \times 1 {0}^{6}$$

### EXAMPLES

### Example 1: ONS-3010/ONS-1045

ONS-3010 (lot #X1302-BDS-O) and ONS-1045(lot #1407104501) were prepared in accordance with the disclosed methods by implementing the reduced digestion time (0.5 hour for trypsin) and by testing the digested peptides with mass spectroscopy using the 98 minute methodology to allow time for peptides to bind to the column. Purified samples of ONS-3010 and ONS-1045, which were analyzed using an 88 minute methodology, were analyzed using the 98 minute methodology, and the results were compared against results obtained using the 88 minute methodology. A summary of the improved sequence coverage is provided in Table 1.

TABLE 1
Sequence Coverage Improvements with 0.5 hour
Trypsin Digestion and 98 Minute UPLC Method

| ONS-3010 | Heavy Chain | Light Chain |
|---|---|---|
| 0.5 hour Trypsin digestion + | 100.00% | 98.60% |
| 98 min instr method | | |
| 1 hour Trypsin digestion + | 95.79% | 97.20% |
| 88 min instr method | | |
| ONS-1045 | Heavy Chain | Light Chain |
| 0.5 hour Trypsin digestion + | 100.00% | 98.60% |
| 98 min instr method | | |
| 1 hour Trypsin digestion + | 96.91% | 98.60% |
| 88 min instr method | | |

Trypsin digested ONS-3010 heavy chain sequence coverage increased from 95.79% to 100% and trypsin digested ONS-1045 heavy chain sequence coverage increased from 96.91% to 100%.

ONS-1045 samples #1407104501 and #1408104502 were analyzed according to the methods of the disclosure using mass spectroscopy implementing the 98 minute UPLC methodology. Samples were frozen at −80° C. and re-injected on the same instrument with the same mobile phases, but with the 98 minute UPLC method, which includes an extra 10 minutes of 0.4 mL/min 100% mobile phase A at the beginning of the gradient. The prior method implemented an 88 minute run without an extra 10 minutes of mobile phase A at the start of the run. According to the results of reinjections, the 98 minute UPLC method increased amino acid sequence coverage of the trypsin digested samples. Furthermore, the peptide CK was detected as cleaved peptide CKVSNK, but was missed in the 88 minute method.

To evaluate the specificity of the assay, matrix, positive control (Humira), and negative control (Rituxan) were used for specificity determination. Matrix showed no hit from target sequence. Positive control had 100% sequence coverage against Adalimumab sequence on both heavy chain (HC) and light chain (LC) after combining the results of the analysis of the trypsin and chymotrypsin digested samples. Negative control showed no sequence coverage on Fab region against Adalimumab sequence. Some peptides were detected in negative control because the constant region amino acid sequence of different IgG1 is the same. Certain figures herein show either sequence coverage results from Proteome Discoverer or total ion chromotograms. The general sequence coverage of each sample is recorded in Table 2.

TABLE 2
Sequence Coverage Summary for Specificity

| | Heavy Chain | | | Light Chain | | |
|---|---|---|---|---|---|---|
| | Try | Chy | Overall | Try | Chy | Overall |
| Matrix | 0.0% | 0.0% | 0.0% | 0.0% | 0.0% | 0.0% |
| ONS-3010 | 100.0% | 100.0% | 100.0% | 98.6% | 89.3% | 100.0% |
| Ref Std. | | | | | | |
| Positive | 98.9% | 84.1% | 100.0% | 98.6% | 89.3% | 100.0% |
| Control - | | | | | | |
| Humira (H35) | | | | | | |
| ONS-3010 | 98.9% | 87.8% | 100.0% | 98.6% | 89.3% | 100.0% |
| Sample | | | | | | |
| Negative | 69.4% | 50.8% | 72.1% | 48.6% | 45.8% | 50.0% |
| Control - | | | | | | |
| Rituxan (M6) | | | | | | |

To evaluate the reproducibility of the assay, an ONS-3010 reference standard was tested in each run and repeated for 3 runs. The heavy chain and light chain sequence coverage of the reference standards and samples from both trypsin and chymotrypsin digestions are recorded in Table 3.

TABLE 3
Reproducibility for ONS-3010 Sequence Method

| | Heavy Chain | | | Light Chain | | |
|---|---|---|---|---|---|---|
| | Try | Chy | Overall | Try | Chy | Overall |
| #1 Ref. Std. | 100.0% | 86.3% | 100.0% | 98.6% | 89.3% | 100.0% |
| #1 Sample | 98.9% | 87.8% | 100.0% | 98.6% | 89.3% | 100.0% |
| #2 Ref. Std. | 99.3% | 81.2% | 100.0% | 98.6% | 89.3% | 100.0% |
| #2 Sample | 99.3% | 86.3% | 100.0% | 98.6% | 89.3% | 100.0% |
| #3 Ref. Std. | 99.3% | 81.2% | 100.0% | 98.6% | 89.3% | 100.0% |
| #3 Sample | 99.3% | 81.2% | 100.0% | 98.6% | 89.3% | 100.0% |

While the disclosure has been described above in conjunction with specific embodiments, alternatives, modifications, permutations, and variations will become apparent to a person of skill in the art in light of the foregoing description. Accordingly, it is intended that the present invention embraces all such alternatives, modifications, and variations as falling within the scope of the claims below.

What is claimed is:

1. A method for determining the biosimilarity of a test protein in relation to a target biologic, the method comprising the steps of:
(a) digesting a first sample of a test protein for a first incubation time using a first protease and digesting a second sample of the test protein for a second incubation time using a second protease, wherein the first sample and the second sample are physically separated;
(b) applying column chromatography and tandem mass spectroscopy to the first sample under conditions sufficient to enhance binding of small peptides to the column, and generating a sequence of the test protein in the first sample;
(c) applying column chromatography and tandem mass spectroscopy to the second sample under conditions sufficient to enhance binding of small peptides to the column, and generating the sequence of the test protein in the second sample, wherein the first sample and second sample are physically separated;
(d) identifying the test protein as biosimilar to the target biologic when the test protein comprises 100% sequence identity to the target biologic; and
(e) identifying the test protein as not biosimilar to the target biologic when the test protein does not comprise 100% sequence identity to the target biologic.

2. The method of claim 1, wherein the monoclonal antibody comprises Adalimumab.

3. The method of claim 1, wherein the first protease is Trypsin.

4. The method of claim 1, wherein the second protease is Chymotrypsin.

5. The method of claim 1, wherein the first digestion period is about 0.1 to about 1.0 hour.

6. The method of claim 5, wherein the first digestion period is about 0.1 to about 0.5 hour.

7. The method of claim 5, wherein the first digestion period is about 0.6 to about 1.0 hour.

8. The method of claim 5, wherein the first digestion period is about 0.5 hours.

9. The method of claim 1, wherein the second digestion period is about 0.1 to about 2.0 hours.

10. The method of claim 9, wherein the second digestion period is about 0.1 to about 1.5 hours.

11. The method of claim 9, wherein the second digestion period is about 1.5 to about 2.0 hours.

12. The method of claim 9, wherein the second digestion period is about 1.5 hours.


SEQUENCE LISTING (26 sequences)

SEQ ID NO: 1 - protein, 451 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 451 aa, Artificial Sequence
  Synthetic Construct
  MOD_RES (22)..(22): Xaa = carboxymethyl
  MOD_RES (96)..(96): Xaa = carboxymethyl
  MOD_RES (148)..(148): Xaa = carboxymethyl
  MOD_RES (204)..(204): Xaa = carboxymethyl
  MOD_RES (222)..(222): Xaa = lysine-loss
  MOD_RES (224)..(224): Xaa = carboxymethyl
  MOD_RES (230)..(230): Xaa = carboxymethyl
  MOD_RES (233)..(233): Xaa = carboxymethyl
  MOD_RES (265)..(265): Xaa = carboxymethyl
  MOD_RES (301)..(301): Xaa = N-linked glycosylation
  MOD_RES (325)..(325): Xaa = carboxymethyl
  MOD_RES (371)..(371): Xaa = carboxymethyl
  MOD_RES (429)..(429): Xaa = carboxymethyl
  MOD_RES (451)..(451): Xaa = lysine-loss
EVQLVESGGGLVQPGRSLRLSXAASGFTFDDYAMHWVRQAPGKGLEWVSAITWNSGHIDYADSVEGRFTISRDNAKNSLYLQMNSLRAEDTAVYYXAKVSYLSTASSLDYWGQGTLVTVSSASTKGPSVFPLAPSSKSTSGGTAALGXLVKDYFPEPVTVSWNSGALTSGVHTFPAVLQSSGLYSLSSVVTVPSSSLGTQTYIXNVNHKPSNTKVDKKVEPXSXDKTHTXPPXPAPELLGGPSVFLFPPKPKDTLMISRTPEVTXVVVDVSHEDPEVKFNWYVDGVEVHNAKTKPREEQYXSTYRVVSVLTVLHQDWLNGKEYKXKVSNKALPAPIEKTISKAKGQPREPQVYTLPPSRDELTKNQVSLTXLVKGFYPSDIAVEWESNGQPENNYKTTPPVLDSDGSFFLYSKLTVDKSRWQQGNVFSXSVMHEALHNHYTQKSLSLSPGX

SEQ ID NO: 3 - protein, 451 aa, Artificial Sequence; identical to SEQ ID NO: 1
  Synthetic Construct

SEQ ID NO: 4 - protein, 451 aa, Artificial Sequence
  Synthetic Construct
  MOD_RES (22)..(22): Xaa = carboxymethyl
  MOD_RES (96)..(96): Xaa = carboxymethyl
  MOD_RES (148)..(148): Xaa = carboxymethyl
  MOD_RES (174)..(174): Xaa = lysine-loss
  MOD_RES (204)..(204): Xaa = carboxymethyl
  MOD_RES (224)..(224): Xaa = carboxymethyl
  MOD_RES (230)..(230): Xaa = carboxymethyl
  MOD_RES (233)..(233): Xaa = carboxymethyl
  MOD_RES (265)..(265): Xaa = carboxymethyl
  MOD_RES (429)..(429): Xaa = carboxymethyl
  MOD_RES (451)..(451): Xaa = lysine-loss
EVQLVESGGGLVQPGRSLRLSXAASGFTFDDYAMHWVRQAPGKGLEWVSAITWNSGHIDYADSVEGRFTISRDNAKNSLYLQMNSLRAEDTAVYYXAKVSYLSTASSLDYWGQGTLVTVSSASTKGPSVFPLAPSSKSTSGGTAALGXLVKDYFPEPVTVSWNSGALTSGVHTXPAVLQSSGLYSLSSVVTVPSSSLGTQTYIXNVNHKPSNTKVDKKVEPKSXDKTHTXPPXPAPELLGGPSVFLFPPKPKDTLMISRTPEVTXVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSXSVMHEALHNHYTQKSLSLSPGX

SEQ ID NO: 5 - protein, 214 aa, Artificial Sequence
  Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 214 aa, Artificial Sequence
  Synthetic Construct
  MOD_RES (23)..(23): Xaa = carboxymethyl
  MOD_RES (88)..(88): Xaa = carboxymethyl
  MOD_RES (134)..(134): Xaa = carboxymethyl
  MOD_RES (194)..(194): Xaa = carboxymethyl
  MOD_RES (214)..(214): Xaa = carboxymethyl
DIQMTQSPSSLSASVGDRVTITXRASQGIRNYLAWYQQKPGKAPKLLIYAASTLQSGVPSRFSGSGSGTDFTLTISSLQPEDVATYYXQRYNRAPYTFGQGTKVEIKRTVAAPSVFIFPPSDEQLKSGTASVVXLLNNFYPREAKVQWKVDNALQSGNSQESVTEQDSKDSTYSLSSTLTLSKADYEKHKVYAXEVTHQGLSSPVTKSFNRGEX

SEQ ID NO: 7 - protein, 214 aa, Artificial Sequence; identical to SEQ ID NO: 5
  Synthetic Construct

SEQ ID NO: 8 - protein, 214 aa, Artificial Sequence
  Synthetic Construct
  MOD_RES (88)..(88): Xaa = carboxymethyl
  MOD_RES (134)..(134): Xaa = carboxymethyl
  MOD_RES (194)..(194): Xaa = carboxymethyl
  MOD_RES (214)..(214): Xaa = carboxymethyl
DIQMTQSPSSLSASVGDRVTITCRASQGIRNYLAWYQQKPGKAPKLLIYAASTLQSGVPSRFSGSGSGTDFTLTISSLQPEDVATYYXQRYNRAPYTFGQGTKVEIKRTVAAPSVFIFPPSDEQLKSGTASVVXLLNNFYPREAKVQWKVDNALQSGNSQESVTEQDSKDSTYSLSSTLTLSKADYEKHKVYAXEVTHQGLSSPVTKSFNRGEX

SEQ ID NO: 9 - protein, 451 aa, Artificial Sequence; identical to SEQ ID NO: 1
  Synthetic Construct

SEQ ID NO: 10 - protein, 451 aa, Artificial Sequence; identical to SEQ ID NO: 2
  Synthetic Construct
  MOD_RES (22)..(22): Xaa = carboxymethyl
  MOD_RES (96)..(96): Xaa = carboxymethyl
  MOD_RES (148)..(148): Xaa = carboxymethyl
  MOD_RES (204)..(204): Xaa = carboxymethyl
  MOD_RES (222)..(222): Xaa = lysine-loss
  MOD_RES (224)..(224): Xaa = carboxymethyl
  MOD_RES (230)..(230): Xaa = carboxymethyl
  MOD_RES (233)..(233): Xaa = carboxymethyl
  MOD_RES (265)..(265): Xaa = carboxymethyl
  MOD_RES (301)..(301): Xaa = N-linked glycosylation
  MOD_RES (325)..(325): Xaa = carboxymethyl
  MOD_RES (371)..(371): Xaa = carboxymethyl
  misc_feature (429)..(429): Xaa can be any naturally occurring amino acid
  MOD_RES (439)..(439): Xaa = carboxymethyl
  MOD_RES (451)..(451): Xaa = lysine-loss

SEQ ID NO: 11 - protein, 451 aa, Artificial Sequence; identical to SEQ ID NO: 1
  Synthetic Construct

SEQ ID NO: 12 - protein, 451 aa, Artificial Sequence; identical to SEQ ID NO: 4
  Synthetic Construct
  MOD_RES (22)..(22): Xaa = carboxymethyl
  MOD_RES (96)..(96): Xaa = carboxymethyl
  MOD_RES (148)..(148): Xaa = carboxymethyl
  MOD_RES (174)..(174): Xaa = lysine-loss
  MOD_RES (204)..(204): Xaa = carboxymethyl
  MOD_RES (224)..(224): Xaa = carboxymethyl
  MOD_RES (230)..(230): Xaa = carboxymethyl
  MOD_RES (233)..(233): Xaa = carboxymethyl
  MOD_RES (265)..(265): Xaa = carboxymethyl
  MOD_RES (429)..(429): Xaa = carboxymethyl
  MOD_RES (451)..(451): Xaa = lysine-loss

SEQ ID NO: 13 - protein, 214 aa, Artificial Sequence; identical to SEQ ID NO: 5
  Synthetic Construct

SEQ ID NO: 14 - protein, 214 aa, Artificial Sequence; identical to SEQ ID NO: 6
  Synthetic Construct
  MOD_RES (23)..(23): Xaa = carboxymethyl
  MOD_RES (88)..(88): Xaa = carboxymethyl
  MOD_RES (134)..(134): Xaa = carboxymethyl
  MOD_RES (194)..(194): Xaa = carboxymethyl
  MOD_RES (214)..(214): Xaa = carboxymethyl

SEQ ID NO: 15 - protein, 214 aa, Artificial Sequence; identical to SEQ ID NO: 5
  Synthetic Construct

SEQ ID NO: 16 - protein, 214 aa, Artificial Sequence; identical to SEQ ID NO: 8
  Synthetic Construct
  MOD_RES (88)..(88): Xaa = carboxymethyl
  MOD_RES (134)..(134): Xaa = carboxymethyl
  MOD_RES (194)..(194): Xaa = carboxymethyl
  MOD_RES (214)..(214): Xaa = carboxymethyl

SEQ ID NO: 17 - protein, 451 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTHGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVPSCSVMHEALHNHYTQKSLSLSPGK

SEQ ID NO: 18 - protein, 451 aa, Artificial Sequence
  Synthetic Construct
  MOD_RES (148)..(148): Xaa = carboxymethyl
  MOD_RES (204)..(204): Xaa = carboxymethyl
  MOD_RES (224)..(224): Xaa = carboxymethyl
  MOD_RES (230)..(230): Xaa = carboxymethyl
  MOD_RES (233)..(233): Xaa = carboxymethyl
  MOD_RES (265)..(265): Xaa = carboxymethyl
  MOD_RES (325)..(325): Xaa = carboxymethyl
  MOD_RES (371)..(371): Xaa = carboxymethyl
  MOD_RES (429)..(429): Xaa = carboxymethyl
  MOD_RES (451)..(451): Xaa = lysine-loss
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTHGPSVFPLAPSSKSTSGGTAALGXLVKDYFPEPVTVSWNSGALTSGVHTFPAVLQSSGLYSLSSVVTVPSSSLGTQTYIXNVNHKPSNTKVDKKVEPKSXDKTHTXPPXPAPELLGGPSVFLFPPKPKDTLMISRTPEVTXVVVDVSHEDPEVKFNWYVDGVEVHNAKTKPREEQYNSTYRVVSVLTVLHQDWLNGKEYKXKVSNKALPAPIEKTISKAKGQPREPQVYTLPPSRDELTKNQVSLTXLVKGFYPSDIAVEWESNGQPENNYKTTPPVLDSDGSFFLYSKLTVDKSRWQQGNVPSXSVMHEALHNHYTQKSLSLSPGX

SEQ ID NO: 19 - protein, 451 aa, Artificial Sequence
  Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVPSCSVMHEALHNHYTQKSLSLSPGK

SEQ ID NO: 20 - protein, 451 aa, Artificial Sequence
  Synthetic Construct
  MOD_RES (22)..(22): Xaa = carboxymethyl
  MOD_RES (148)..(148): Xaa = carboxymethyl
  MOD_RES (174)..(174): Xaa = lysine-loss
  MOD_RES (265)..(265): Xaa = carboxymethyl
  MOD_RES (429)..(429): Xaa = carboxymethyl
  MOD_RES (451)..(451): Xaa = lysine-loss
EVQLVESGGGLVQPGRSLRLSXAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGXLVKDYFPEPVTVSWNSGALTSGVHTXPAVLQSSGLYSLSSVVTVPSSSLGTQTYICNVNHKPSNTKVDKKVEPKSCDKTHTCPPCPAPELLGGPSVFLFPPKPKDTLMISRTPEVTXVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVPSXSVMHEALHNHYTQKSLSLSPGX

SEQ ID NO: 21 - protein, 214 aa, Artificial Sequence; identical to SEQ ID NO: 5
  Synthetic Construct

SEQ ID NO: 22 - protein, 214 aa, Artificial Sequence
  Synthetic Construct
  MOD_RES (134)..(134): Xaa = carboxymethyl
  MOD_RES (194)..(194): Xaa = carboxymethyl
  MOD_RES (214)..(214): Xaa = carboxymethyl
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVXLLNNFYPREAKVQWKVDNALQSGNSQESVTEQDSKDSTYSLSSTLTLSKADYEKHKVYAXEVTHQGLSSPVTKSFNRGEX

SEQ ID NO: 23 - protein, 214 aa, Artificial Sequence; identical to SEQ ID NO: 5
  Synthetic Construct

SEQ ID NO: 24 - protein, 214 aa, Artificial Sequence; identical to SEQ ID NO: 22
  Synthetic Construct
  MOD_RES (134)..(134): Xaa = carboxymethyl
  MOD_RES (194)..(194): Xaa = carboxymethyl
  MOD_RES (214)..(214): Xaa = carboxymethyl

SEQ ID NO: 25 - protein, 214 aa, Artificial Sequence; identical to SEQ ID NO: 5
  Synthetic Construct

SEQ ID NO: 26 - protein, 451 aa, Artificial Sequence; identical to SEQ ID NO: 1
  Synthetic Construct